(19) United States
(12) Patent Application Publication
(10) Pub. No.: US 2020/0094005 A1
(43) Pub. Date: Mar. 26, 2020

(54) RADIO FREQUENCY POWERED AIRWAY PRESSURE SUPPORT DEVICE CROSS-REFERENCE TO RELATED APPLICATIONS

(71) Applicant: KONINKLIJKE PHILIPS N.V., Eindhoven (NL)

(72) Inventor: RICHARD DANIEL SOFRANKO, MONROEVILLE, PA (US)

(21) Appl. No.: 16/577,918

(22) Filed: Sep. 20, 2019

Related U.S. Application Data
(60) Provisional application No. 62/734,328, filed on Sep. 21, 2018.

(51) Int. Cl. A61M 16/00; A61M 16/06; A61M 16/20

(52) U.S. Cl. CPC A61M 16/022; A61M 16/0066; A61M 16/0633; A61M 16/208; A61M 16/0611; A61M 2205/3592; A61M 2230/50; A61M 2205/3334; A61M 2205/3344; A61M 2205/3368; A61M 2205/3365; A61M 2230/43; A61M 2230/42; A61M 2205/3584

(57) ABSTRACT

A patient interface device comprising a cushion and a frame and housing member coupled to the cushion. The frame and housing member includes a pressure generating system structured to generate a flow of breathing gas and is in fluid communication with the cushion. The frame and housing member also includes an antenna and RF energy harvesting circuitry coupled to the antenna, wherein the antenna is structured to receive RF energy and provide the RF energy to the RF energy harvesting circuitry, and wherein the RF energy harvesting circuitry is structured to convert the RF energy into usable energy for powering the pressure generating system.

### CROSS-REFERENCE TO RELATED APPLICATIONS

This patent application claims the priority benefit under 35 U.S.C. § 119(e) of U.S. Provisional Application No. 62/734,328, filed on Sep. 21, 2018, the contents of which are herein incorporated by reference.

### BACKGROUND OF THE INVENTION

### 1. Field of the Invention

The present invention pertains to airway pressure support devices, and, in particular, to an airway pressure support device that includes a patient interface device having an integrated gas flow generator that is powered by radio frequency (RF) energy.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion on the face of the patient. The mask component may be, without limitation, a nasal mask that covers the patient's nose, a nasal/oral mask that covers the patient's nose and mouth, or a full face mask that covers the patient's face. The patient interface device is typically secured to the patient's head by a headgear component. Traditionally, the patient interface device is connected to a separately housed pressure/flow generating device, such as a blower unit, by way of a gas delivery tube or conduit. The pressure/flow generating device generates a flow of positive pressure breathing gas that is delivered to the airway of the patient through the patient interface device for purposes of “splinting” the airway as described above.

A frequent complaint of the users of such pressure support therapies is the discomfort that is associated with sleeping in bed with the gas delivery tube or conduit that connects the patient interface device to the housing that includes the pressure/flow generating device. This discomfort discourages the regular use of such devices, and therefore increases the risk of worsening OSA.

### SUMMARY OF THE INVENTION

Accordingly, it is an object of the present invention to provide a pressure support device that overcomes the shortcomings of conventional pressure support devices. This object is achieved according to one embodiment of the present invention by providing a patient interface device for delivering a flow of breathing gas to an airway of a patient that a cushion an a frame and housing member directly coupled to the cushion. The frame and housing member includes a pressure generating system provided within the frame and housing member and structured to generate the flow of breathing gas, the pressure generating system being in fluid communication with the cushion, an antenna, and radio frequency (RF) energy harvesting circuitry provided within the frame and housing member and coupled to the antenna. The antenna is structured to receive radio frequency (RF) energy and provide the RF energy to the RF energy harvesting circuitry, and the RF energy harvesting circuitry is structured to convert the RF energy into usable energy for powering the pressure generating system.

In another embodiment, the patient interface device is part of an airway pressure support system that also includes an RF base unit spaced from the patient interface device. The RF base unit in this embodiment is structured and configured to generate the radio frequency (RF) energy received by the patient interface device for powering the patient interface device.

In still another embodiment, a method of generating a flow of breathing gas to be delivered to an airway of a patient is provided. The method includes generating radio frequency (RF) energy in an RF base unit and transmitting the RF energy from the RF base unit, receiving the RF energy in a patient interface device spaced from the RF base unit, the patient interface device including a cushion and a frame and housing member directly coupled to the cushion, the frame and housing member including a pressure generating system provided within the frame and housing member and being in fluid communication with the cushion, converting the RF energy into usable energy, such as, without limitation, a DC voltage, and powering the pressure generating system using the usable energy to generate the flow of breathing gas and provide the flow of breathing gas to the cushion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram showing an RF powered airway pressure support system according to one particular, non-limiting exemplary embodiment of the disclosed concept;

FIG. 2 is a schematic diagram of a frame and housing member of a patient interface device shown in FIG. 1 according to one particular, non-limiting exemplary embodiment, including the various components housed therein; and

FIG. 3 is a schematic diagram of an RF base unit of the system shown in FIG. 1 according to one particular, non-limiting exemplary embodiment, including the various components housed therein.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As described in greater detail herein in connection with various particular embodiments, the disclosed concept provides a built-in blower patient interface device (e.g., CPAP mask) that is wirelessly powered by RF energy, such as RF energy transmitted by an associated RF transmitter that is spaced from and not directly coupled or connected to the patient interface device. In addition to transmitting power, such an RF transmitter may also communicate with the built-in blower patient interface device over a wireless network for purposes of controlling the delivery of therapy and/or gathering sleep related or other data over a wireless network. This wireless powering and communication scheme can be done with a certain physical distance between the two devices which separates the patient from the transmitter.

In the exemplary embodiment, the patient interface device (e.g., mask) includes a pressure/flow generating device (e.g., a blower unit) that is built in to the patient interface device (e.g., housed within a housing formed by a frame member of the patient interface device). The patient interface device may also have one or more sensors for detecting certain quantifiable metrics, including, without limitation, one or more of flow rate, humidity, pressure, temperature, and blower fan RPM, including parameters of the patient, such as SpO2, breath rate, body temperature, etc. The patient interface device will also include an antenna device for receiving data/power from the RF transmitter over a wireless network. This antenna device may also be used for sending data generated by the above-described the sensors to the RF transmitter. In addition, the patient interface device will further include an integrated RF harvesting device that converts radio frequency (RF) energy received by the antenna (e.g., from the RF transmitter and/or from the ambient environment) into appropriate AC or DC current for powering the built-in pressure/flow generating device. The patient interface device also includes an accompanying headgear for securing it on the patient's face.

The components described above are, in the exemplary embodiment, designed to fit in one compact housing that is part of the patient interface device (e.g., a combination housing/frame member that is connected to a soft, flexible sealing cushion). In one embodiment, such a compact housing can be designed as an addition to a existing cushion (possibly via snap-fit or magnet fittings). In an alternate embodiment, the disclosed patient interface device can be designed wholesale per cushion type (e.g., full-face, full-face under the nose, nasal, nasal pillows).

In addition, the details for implementing the disclosed RF harvesting device and the set-up between the untethered receiver (i.e., the patient interface device) and the transmitter of RF energy (e.g., the RF transmitter) may, in one particular embodiment, be as described in U.S. Pat. Nos. 9,021,277 and 9,107,579, the disclosures of which are incorporated herein by reference.

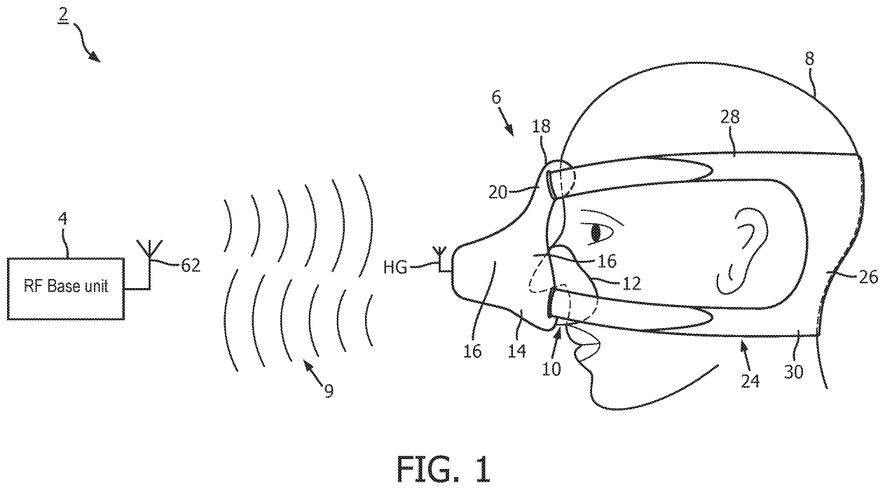

FIG. 1 is a schematic diagram showing an RF powered airway pressure support system 2 according to one particular, non-limiting exemplary embodiment of the disclosed concept which is operated within an environment, such as a bedroom, of the user of airway pressure support system 2. Referring to FIG. 1, airway pressure support system 2 includes an RF base unit 4 and a patient interface device 6, each of which is described in greater detail herein. RF base unit 4 is structured to rest on a structure provided within the environment, such as, without limitation, a piece of furniture like a nightstand that is in close proximity to the bed of the user of airway pressure support system 2. Patient interface device 6 is structured to be worn by or otherwise attached to a patient 8. As described in greater detail herein, patient interface device 6 is structured to generate and communicate a flow of breathing gas to the airway of patient 8 in order to provide airway pressure support therapy to patient 8. As seen, patient interface device 6 is spaced from and not directly/physically coupled or connected to the RF base unit 4. Rather, as described below, patient interface device 6 and RF base unit 4 are operatively coupled to one another only through an air interface by way of a wireless communications network (i.e., the two devices are not physically in contact with one another).

Furthermore, as described in detail herein, airway pressure support system 2 is provided with functionality that enables patient interface device 6 to be wirelessly powered by RF energy that is generated by RF base unit 4 and/or that may be present in the ambient environment surrounding patient interface device 6. In addition, in the non-limiting exemplary embodiment, airway pressure support system 2 is further provided with functionality that enables RF base unit 4 and patient interface device 6 to wirelessly communicate with one another over a wireless network (e.g., so that data related to control of patient interface device 6 may be communicated from our base unit 4 to patient interface device 6 and/or so that data relating to operation of patient interface device 6 and metrics measured thereby may be communicated from patient interface device 6 to RF base unit 4). More specifically, in the exemplary embodiment, RF base unit 4 and patient interface device 6 are configured to communicate with one another via and within the operational range of a wireless personal area network (PAN) 9 shown schematically in FIG. 1. Similarly, in the exemplary embodiment, RF base unit 4 is configured to transmit a sufficient amount of power for powering patient interface device 6 as described herein via and within the operational range of PAN 9.

In the exemplary embodiment, patient interface device 6 includes a patient sealing assembly 10, which in the illustrated embodiment is a nasal mask. However, other types of patient sealing assemblies, such as, without limitation, a nasal/oral mask, a nasal cushion, nasal pillows, or a full face mask, which facilitate the delivery of the flow of breathing gas to the airway of a patient may be substituted for patient sealing assembly 10 while remaining within the scope of the present invention.

Patient sealing assembly 10 includes a cushion 12 coupled to a frame and housing member 14. In the illustrated embodiment, cushion 12 is defined from a unitary piece of soft, flexible, cushiony, elastomeric material, such as, without limitation, silicone, an appropriately soft thermoplastic elastomer, a closed cell foam, or any combination of such materials. Also in the illustrated embodiment, frame and housing member 14 is structured to house various components described in detail below, and is made of a rigid or semi-rigid material, such as, without limitation, an injection molded thermoplastic or silicone. Frame and housing member 14 includes a faceplate portion 16 to which cushion 12 is fluidly attached, and a forehead support member 18 that is coupled to faceplate portion 16 by a connecting member 20. A forehead cushion 22 is coupled to the rear of forehead support member 18. In the exemplary embodiment, forehead cushion 22 is made of a material that is similar to the material of cushion 12. Patient interface device 10 also includes a headgear component 24 for securing patient interface device 10 to the head of patient 8. Headgear component 24 includes a back member 26, upper strap members 28, and lower strap members 30. In the exemplary embodiment, upper strap members 28 and lower strap members 30 each include a hook and loop fastening system, such as VELCRO®, provided on the end thereof to allow headgear component 24 to be secured in a known manner. It will be understood that the described hook and loop fastening arrangement is meant to be exemplary only, and that other selectively adjustable fastening arrangements are also possible within the scope of the present invention.

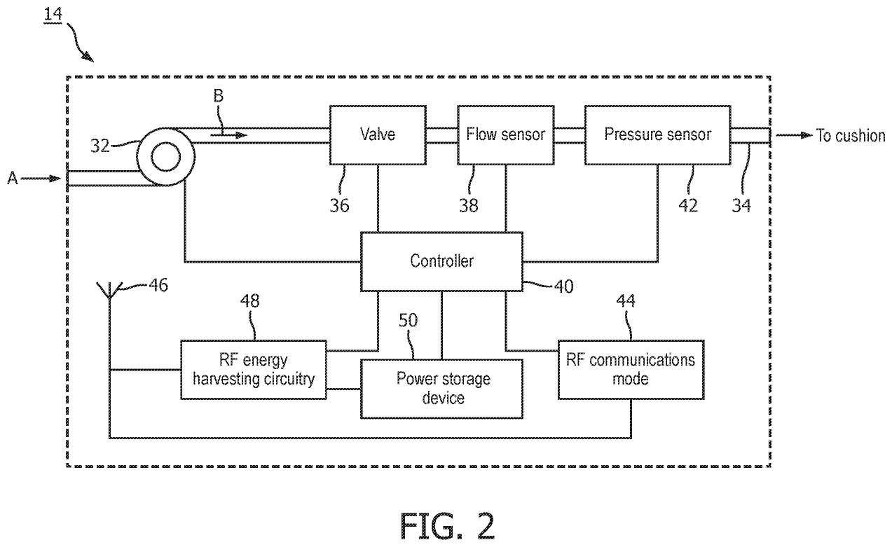

FIG. 2 is a schematic diagram of frame and housing member 14 according to one particular, non-limiting exemplary embodiment, including the various components housed therein. As seen in FIG. 2, frame and housing member 14 includes a gas flow generator 42 (such as a conventional blower unit including a fan) that receives breathing gas, generally indicated by arrow A, from the ambient atmosphere (e.g., through a vent or opening (not shown) provided in frame and housing member 14) and generates a flow of breathing gas therefrom for delivery to an airway of patient 8 at relatively higher and lower pressures, i.e., generally equal to or above ambient atmospheric pressure. In the exemplary embodiment, gas flow generator 32 is capable of providing a flow of breathing gas ranging in pressure from 3-30 cmH2O. The pressurized flow of breathing gas from gas flow generator 32, generally indicated by arrow B, is delivered via a delivery conduit 34 to cushion 12 to communicate the flow of breathing gas to the airway of patient 8. In addition, an exhaust vent (not shown) is provided in patient interface device 6 for venting exhaled gases from patient interface device 6. It should be understood that the exhaust vent can have a wide variety of configurations depending on the desired manner in which gas is to be vented from patient interface device 6.

In the illustrated embodiment, frame and housing member 14 includes a pressure controller in the form of a valve 36 provided in delivery conduit 34. Valve 36 controls the pressure of the flow of breathing gas from gas flow generator 32 that is delivered to patient 8. For present purposes, gas flow generator 32 and valve 36 are collectively referred to as a pressure generating system because they act in concert to control the pressure and/or flow of gas delivered to patient 8. However, it should be apparent that other techniques for controlling the pressure of the gas delivered to patient 8, such as varying the blower speed of gas flow generator 32, either alone or in combination with a pressure control valve, are contemplated by the present invention. Thus, valve 36 is optional depending on the technique used to control the pressure of the flow of breathing gas delivered to patient 8. If valve 36 is eliminated, the pressure generating system corresponds to gas flow generator 32 alone, and the pressure of gas in delivery conduit 34 is controlled, for example, by controlling the motor speed of gas flow generator 32.

Frame and housing member 14 further includes a flow sensor 38 that measures the flow of the breathing gas within delivery conduit 34. In the particular embodiment shown in FIG. 2, flow sensor 38 is interposed in line with delivery conduit 34 downstream of valve 36. Flow sensor 38 generates a flow signal, Q_{MEASURED}, that is provided to a controller 40 provided in frame and housing member 14 and is used by controller 40 to determine the flow of gas at patient 8 (Q_{PATIENT}). Techniques for calculating Q_{PATIENT} based on Q_{MEASURED} are well known, and take into consideration the pressure drop of the patient circuit, known leaks from the system, i.e., the intentional exhausting of gas from the circuit as described herein, and unknown (unintentional) leaks from the system, such as leaks at the mask/patient interface. The present invention contemplates using any known or hereafter developed technique for calculating total leak flow Q_{LEAK}, and using this determination in calculating Q_{PATIENT} based on Q_{MEASURED} (and for other purposes as described elsewhere herein). Examples of such techniques are taught by U.S. Pat. Nos. 5,148,802; 5,313,937; 5,433,193; 5,632,269; 5,803,065; 6,029,664; 6,539,940; 6,626,175; and 7,011,091, the contents of each of which are incorporated by reference into the present invention.

In the illustrated embodiment, frame and housing member 14 also further includes a pressure sensor 42 that measures the pressure of the breathing gas within delivery conduit 34. In the particular embodiment shown in FIG. 2, pressure sensor 42 is interposed in line with delivery conduit 34 downstream of valve 36. Pressure sensor 38 generates a pressure signal that is provided to controller 40.

Additional sensors, in place of or in addition to flow sensor 38 and pressure sensor 42, may also be provided within frame and housing member 14 and coupled to controller 40. Such additional sensors may include, without limitation, a humidity sensor, a temperature sensor, and/or a blower fan RPM sensor.

Controller 40 includes a processing portion which may be, for example, a microprocessor, a microcontroller, an application specific integrated circuit (ASIC) or some other suitable processing device. Controller 40 also includes a memory portion, such as a random access memory and/or a read only memory, that may be internal to the processing portion or operatively coupled to the processing portion and that provides a storage medium for data and software executable by the processing portion for controlling the operation of patient interface device 6.

As seen in FIG. 2, frame and housing member 14 further includes an RF communications module 44 which is operatively coupled to an antenna 46 and to controller 40. RF communications module 44, such as an RF radio or similar device operating at any appropriate frequency, is structured and configured to generate an RF signal to be wirelessly transmitted by antenna 46 over PAN 9 to RF base unit 4. The signal generated by RF communications module 44 is, in the exemplary embodiment, a data signal generated by controller 40 that includes information relating to and/or based upon the parameters measured by flow sensor 38 and/or pressure sensor 42 (or any other sensor described herein) and/or information relating to operation of patient interface device 6, such as operation of gas flow generator 32 (e.g., the operational speed thereof). The RF communications module is structured and configured to communicate with PAN 9 using any suitable wireless protocol such as, without limitation, Wi-Fi®, or Bluetooth®. Alternatively, RF communications module 44 may comprise load modulation circuitry that is structured to modulate an RF carrier signal sent from an external source, such as RF base unit 4, in order to communicate the data signal generated by controller 40 to the external source. Antenna 46 may be any suitable antenna such as, without limitation, a dipole antenna, monopole antenna, a patch antenna, or a multi-band antenna.

In the exemplary embodiment, RF base unit 4 and patient interface device 6 are structured and configured to communicate with one another wirelessly using either the near-field region or the far-field region. The RFID Handbook by the author Klaus Finkenzeller defines the inductive coupling or near-field region as distance between the transmitter and receiver of less than 0.16 times lambda where lambda is the wavelength of the RF wave, and the far-field region as distances greater than 0.16 times lambda, and those definitions shall be used herein.

As also seen in FIG. 2, frame and housing member 14 includes RF energy harvesting circuitry 48 that is coupled to antenna 46. RF energy harvesting circuitry 48 is structured to receive RF energy from an external source, such as RF base unit 4, via antenna 46 and harvest energy therefrom by converting (e.g., rectifying) the received RF energy into usable energy, such as a DC or AC voltage. The usable energy (e.g. a DC voltage) is then used to power the other components of patient interface device 6 described above either directly or after being stored in a power storage device 50 (e.g., a rechargeable battery). RF energy harvesting circuitry 48 may include antenna matching circuitry, rectifying circuitry, voltage transforming circuitry, and/or other performance optimizing circuitry. The rectifying circuitry (which applies to conversion of RF to DC) may include a diode(s), a transistor(s), or some other rectifying device or combination. Examples of suitable rectifying circuitry include, but are not limited to, half-wave, full-wave, and voltage doubling circuits. U.S. Pat. No. 6,615,074, incorporated by reference, herein, shows numerous examples of RF energy harvesting circuitry that can be used to implement the function just described.

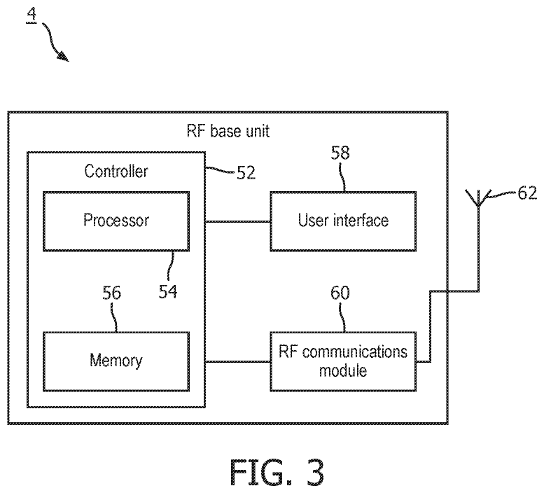

FIG. 3 is a schematic diagram of RF base unit 4 according to one particular, non-limiting exemplary embodiment, including the various components housed therein. As seen in FIG. 3, RF base unit 4 includes a controller 52 that includes a processor 54, which may be, for example, a microprocessor, a microcontroller, an application specific integrated circuit (ASIC) or some other suitable processing device. Controller 52 also includes a memory 56, such as a random access memory and/or a read only memory, that may be internal to the processor 54 or operatively coupled to processor 54 and that provides a storage medium for data and software executable by processor 54 for controlling the operation of RF base unit 4. RF base unit 4 also includes a user interface 58 (which enables information to be input into and output from RF base unit 4). User interface 58 may include a display, a keyboard, a touchscreen, or some combination thereof. As seen in FIG. 3, RF base unit 4 further includes an RF communications module 60 which is operatively coupled to an antenna 62 and to controller 52. RF communications module 60 is structured and configured to generate an RF signal to be wirelessly transmitted by antenna 62 over PAN 9 to patient interface device 6 using any of the wireless protocols described herein. In the exemplary embodiment, the RF signal generated by RF communications module includes at least a power signal and may include a data signal with a power component.

In operation, a user will strap patient interface device 6 to his/her face me a headgear component 24. The user then turns on RF base unit 4, and RF base unit 4 begins generating and transmitting RF energy from antenna 62 to PAN 9. The transmitted RF energy is received via PAN 9 by antenna 46 of patient interface device 10 and is converted to usable energy as described herein. That usable energy is then used by patient interface device 6 to power the components thereof. In particular, the energy is used to power gas flow generator 32 to enable gas flow generator 32 to generate a flow of breathing gas that is delivered to the airway of patient 8 through cushion 12 as described herein in order to provide pressure support therapy to patient 8. In addition, RF base unit may wirelessly communicate data (e.g., commands) to patient interface device 10 via PAN 9 for controlling operation of patient interface device 10, including pressure levels to be generated by the pressure generating system of patient interface device 10. In particular, such data will be transmitted by antenna 62 of RF base unit 4 and received by antenna 46 of patient interface device 6. The data signal will then be provided to controller 40 through RF communications module 44 so that controller 40 may then use the information in the data signal to control the operation of patient interface device 6, including control of the pressure generating system patient based device. In addition, a data signal generated by controller 40 based on the outputs of flow sensor 38, pressure sensor 42 and/or any other sensor described herein may be provided to RF communications module 44 for transmission via antenna 46 to PAN 9. That signal may then be received by antenna 62 of RF base 4 for use in (e.g., analysis of) or storage by controller 52 of RF base unit 4.

In the illustrated, non-limiting exemplary embodiment, airway pressure support system 2 essentially functions as a CPAP pressure support system, and, therefore, includes all of the capabilities necessary in such systems in order to provide appropriate CPAP pressure levels to patient 8. This includes receiving the necessary parameters, via input commands, signals, instructions or other information in patient interface device 6 from RF base unit 4 as described above, for providing appropriate CPAP pressure, such as maximum and minimum CPAP pressure settings. It should be understood that this is meant to be exemplary only, and that other pressure support methodologies, including, but not limited to, BiPAP AutoSV, AVAPS, Auto CPAP, and BiPAP Auto, are within the scope of the present invention.

In the exemplary embodiment, RF base unit 4 and patient interface device 6 are structured and configured to communicate with one another wirelessly using either the near-field region or the far-field region. The RFID Handbook by the author Klaus Finkenzeller defines the inductive coupling or near-field region as distance between the transmitter and receiver of less than 0.16 times lambda where lambda is the wavelength of the RF wave, and the far-field region as distances greater than 0.16 times lambda, and those definitions shall be used herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A patient interface device for delivering a flow of breathing gas to an airway of a patient, comprising:
a cushion; and
a frame and housing member directly coupled to the cushion, the frame and housing member including:
a pressure generating system provided within the frame and housing member and structured to generate the flow of breathing gas, the pressure generating system being in fluid communication with the cushion;
an antenna; and
radio frequency (RF) energy harvesting circuitry provided within the frame and housing member and coupled to the antenna, wherein the antenna is structured to receive RF energy and provide the RF energy to the RF energy harvesting circuitry, and wherein the RF energy harvesting circuitry is structured to convert the RF energy into usable energy for powering the pressure generating system.

2. The patient interface device according to claim 1, wherein the usable energy is a DC voltage and wherein the RF energy harvesting circuitry is structured to convert the RF energy into the DC voltage.

3. The patient interface device according to claim 1, further comprising an RF communications module coupled to the antenna, wherein the RF communications module is structured and configured to generate an RF data signal and provide the RF data signal to the antenna for wireless transmission from the patient interface device.

4. The patient interface device according to claim 3, further comprising a controller coupled to the RF energy harvesting circuitry and the RF communications module, the controller being structured and configured to be powered by the usable energy generated by the RF energy harvesting circuit, the controller being further structured and configured to provide information to the RF communications module for enabling the RF communications module to generate the RF data signal.

5. The patient interface device according to claim 4, further comprising a number of sensors coupled to the controller, wherein the information provided by the controller to the RF communications module for generation of the RF data signal is based on one or more signals received from the number of sensors.

6. The patient interface device according to claim 3, further comprising a controller coupled to the RF energy harvesting circuitry and the RF communications module, the controller being structured and configured to be powered by the usable energy generated by the RF energy harvesting circuit, the controller being further structured and configured to receive a control signal from the RF communications module, wherein the controller is further structured and configured to control operation of the pressure generating device based on the control signal.

7. The patient interface device according to claim 1, wherein the pressure generating system includes a gas flow generator powered by the usable energy.

8. A pressure support system, comprising the patient interface device comprising:
a cushion; and
a frame and housing member directly coupled to the cushion, the frame and housing member including:
a pressure generating system provided within the frame and housing member and structured to generate the flow of breathing gas, the pressure generating system being in fluid communication with the cushion,
an antenna, and
radio frequency (RF) energy harvesting circuitry provided within the frame and housing member and coupled to the antenna, wherein the antenna is structured to receive RF energy and provide the RF energy to the RF energy harvesting circuitry, and wherein the RF energy harvesting circuitry is structured to convert the RF energy into usable energy for powering the pressure generating system; and
an RF base unit spaced from the patient interface device, the RF base unit being structured and configured to generate the RF energy received by the patient interface device for powering the patient interface device.

9. The pressure support system according to claim 9, wherein the RF base unit includes a second controller, a second RF communications module coupled to the second controller, and a second antenna coupled to the second RF communications module, wherein the second controller stores information for controlling operation of the pressure generating system of the patient interface device including pressure levels to be generated thereby, and wherein the second RF communications module is structured and configured to generate and wirelessly transmit through the second antenna one or more RF control signals for controlling operation of the pressure generating system based on the information stored by the second controller, wherein the antenna and the RF communications module of the patient interface device are structured and configured to receive the one or more RF control signals, and wherein the controller of the patient interface device is further structured and configured to control operation of the pressure generating device based on the received one or more RF control signals.

10. The pressure support system according to claim 9, wherein the RF base unit includes a second RF communications module and a second antenna coupled to the second RF communications module, and wherein the second RF communications module is structured and configured to generate and wirelessly transmit through the second antenna the RF energy.

11. A method of generating a flow of breathing gas to be delivered to an airway of a patient, comprising:
generating radio frequency (RF) energy in an RF base unit and transmitting the RF energy from the RF base unit;
receiving the RF energy in a patient interface device spaced from the RF base unit, the patient interface device including a cushion and a frame and housing member directly coupled to the cushion, the frame and housing member including a pressure generating system provided within the frame and housing member and being in fluid communication with the cushion;
converting the RF energy into usable energy;
powering the pressure generating system using the usable energy to generate the flow of breathing gas and provide the flow of breathing gas to the cushion.

12. The method according to claim 12, the patient interface device including an antenna and RF energy harvesting circuitry provided within the frame and housing coupled to the antenna, wherein the antenna is structured to receive RF energy and provide the RF energy to the RF energy harvesting circuitry, and wherein the RF energy harvesting circuitry is structured to convert the RF energy into the usable energy.

13. The method according to claim 11, further comprising generating an RF data signal and transmitting the RF data signal from the patient interface device, and receiving the RF data signal in the RF base unit.

14. The method according to claim 11, wherein the patient interface device further comprises a number of sensors, wherein the RF data signal is based on one or more signals generated by the number of sensors.

15. The method according to claim 13, further comprising transmitting from the RF base unit one or more RF control signals for controlling operation of the pressure generating system including pressure levels to be generated thereby, receiving in the patient interface device one or more RF control signals, and controlling operation of the pressure generating device based on the received one or more RF control signals.
